(19) United States
(12) Patent Application Publication
(10) Pub. No.: US 2017/0025028 A1
(43) Pub. Date: Jan. 26, 2017

(54) ACTIGRAPHY BASED BIOLOGICAL RHYTHM MODIFICATION METHODS AND SYSTEMS THAT RESULT IN A GREATER EFFICACY OF APPLIED MEDICAL TREATMENT TO A PATIENT

(71) Applicant: William J. M. HRUSHESKY, West Orange, NJ (US)

(72) Inventor: William J. M. HRUSHESKY, West Orange, NJ (US)

(73) Assignee: Rhythmalytics LLC, West Orange, NJ (US)

(21) Appl. No.: 15/206,888

(22) Filed: Jul. 11, 2016

Related U.S. Application Data
(60) Provisional application No. 62/196,035, filed on Jul. 23, 2015.

(51) Int. Cl. G09B 5/12; A61B 5/00; G02C 7/10; G09B 5/04; G09B 19/00; A61B 5/11; A61B 5/0205

(52) U.S. Cl. CPC G09B 5/125; A61B 5/11; A61B 5/4857; A61B 5/486; A61B 5/7475; A61B 5/742; A61B 5/0022; A61B 5/02055; A61B 5/7405; A61B 5/7207; A61B 5/4306; G09B 5/04; G09B 19/00; G02C 7/104; A61B 2562/0219; A61B 2560/0242; A61B 2560/0214; A61B 5/024

(57) ABSTRACT

There is a monitoring system that can be employed by any number of users that monitors their physiological state and environmental surroundings. Based on this information, the system provides recommendations for the changing and/or modification of certain behaviors. These modified and/or changed behaviors are directly related to the perceived biological rhythm disruption registered by the system. By modifying and thereby tuning these biological rhythm disruptions, users can help prevent disease, improve quality of life, and alleviate certain conditions.

### CLAIM OF PRIORITY

This application claims priority to U.S. Application 62/196,035 filed on Jul. 23, 2015, the contents of which are herein fully incorporated by reference in its entirety.

### FIELD OF THE EMBODIMENTS

The field of the present invention and its embodiments relate to treatments for various disease states involving fundamental modifications to at least one biological rhythm associated with a patient. In particular, treatment is effectuated via an actigraphic monitoring system paired with dedicated software to finely tune at least one biological rhythm of the patient thereby increasing the efficacy of applied medical treatments.

### BACKGROUND OF THE EMBODIMENTS

Innate biological rhythms are present in a wide host of organisms including animals, plants, fungi, and cyanobacteria. The formal study of these biological temporal rhythms, such as daily, tidal, weekly, seasonal, and annual rhythms, is known as chronobiology. These rhythms are vitally important to the everyday functioning and overall health of the organism in question. At times, it is prudent to treat a particular disease state or other malady with tuning and/or modification of one or more of these biological rhythms. Chronotherapy refers to the use of circadian or other biological rhythmic cycles in the application of therapy to a subject both human and non-human. Indeed, such biological rhythms in sickness and in health are critical factors, hence effective treatment must work with the biological clocks of the subject, not against them.

There is little doubt that disrupted circadian rhythm time structures are involved in, not only the initiation, but also the promotion of neoplastic disease. Studies have shown that nocturnal exposure to light causing the disruption of circadian functionality equates to an increased tumor burden experienced by the individual receiving such disruption. In addition, untreated cancer patients suffer from a number of maladies that are related to circadian rhythm disruption such as but not limited to: poor sleeping patterns, depressed mood/anxiety, fatigue, and eating patterns disruptions. However, treatment of circadian rhythm disruption can lead to improved states of the subject.

For example, melatonin administration has been shown to produce a variety of benefits amongst cancer patients. Melatonin is a daily time-keeping hormone produced by the pineal gland and secreted during the night. This secretion is driven by the endogenous circadian pacemaker in the suprachiasmatic nuclei of the anterior hypothalamus. The application of melatonin, as previously noted, can result in a reduction in the frequency and/or severity of treatment related side effects including myelosupression, neurotoxicity, nephrotoxicity, cardiotoxicity, and asthenia. Melatonin has also been shown to relive anxiety, improve sleep quality, and diminish cachexia with progressing cancer.

Further, the monitoring of a woman's menstrual cycle may be paramount in timing the scheduling of cancer surgeries, mammography, biopsies, etc. A mammogram should typically be scheduled to diminish the chances of missing cancer present in a breast. The cancer may be missed or not readily visible due to the fluctuations in the density of breast tissue throughout the menstrual cycle. Typically, breast density, tenderness, and swelling are decreasing most rapidly during the “follicular’” phase of that particular cycle.

In turn, biopsies of the breast tissues should be done at an opposing time in the menstrual cycle, typically about 14 to about 21 days after the first day of the last menstrual period. This time frame is likely to suppress cancer growth and spreading during surgical intervention. Thus, this “luteal” phase, where little cell division is occurring in the follicle, decreases the chance of adverse cancer outcomes if the wounding of the biopsy is performed. The application of chemotherapy and other treatment considerations can also be made with particular attention to the menstrual cycle of a particular woman. Such considerations may result in up to about 26% increase in the curative properties of a surgical resection performed with the above menstrual phases in mind.

Consequently, therapeutic manipulations of the circadian clock (i.e. chronotherapies) is a powerful tool to improve cancer patients (and others) quality of life, even perhaps leading to higher survival rates. As has been previously shown in the literature, chronotherapy is significantly less toxic and more effective than constant rate infusion. Such results support the concept of temporal selectively of cancer chemotherapy treatment.

Some attempts to employ chronotherapies in the treatment of disease have been implemented, albeit with tepid results. Typically such methodologies rely on point measurements and spot treatments to alleviate symptoms of circadian disruption and/or reset the circadian clock. Such applications include the uptake of melatonin for insomnia and phototherapies for seasonal affective disorders.

Thus, there is a need to take advantage of such non-invasive forms of treatment. It is desirable to allow people to identify and correct disruptions to their biological rhythms to reduce the risk of disease and improve their quality of life. Such a system should allow for the real-time monitoring of a number of biological rhythms and factors contributing to the cyclical disruption thereby allowing for recommendations to correct and entrain natural healthy biorhythms. The present invention and its embodiments meets and exceeds these objectives.

### Review of Related Technology:

U.S. Pat. No. 8,741,336 pertains to systems and methods for longevity, anti-aging, fatigue management, obesity, weight loss, weight management, delivery of nutraceuticals, and treating hyperglycemia, Alzheimer's disease, sleep disorders, Parkinson's disease, Attention Deficit Disorder and nicotine addiction involve synchronizing and tailoring the administration of nutraceuticals, medications and other substances (for example, stimulants) in accordance with the body's natural circadian rhythms, meal times and other factors. Improved control of blood glucose levels, extended alertness, and weight control, and counteracting of disease symptoms when they are at their worst are possible. An automated, pre-programmable transdermal administration system is used to provide pulsed doses of medications, pharmaceuticals, hormones, neuropeptides, anorexigens, pro-drugs, stimulants, plant extracts, botanicals, nutraceuticals, cosmeceuticals, phytochemicals, phytonutrients, enzymes, antioxidants, essential oils, fatty acids, minerals, vitamins, amino acids, coenzymes, or other physiological active ingredient or precursor. The system can utilize a pump, pressurized reservoir, a system for removing depleted carrier solution, or other modulated dispensing actuator, in conjunction with porous membranes or micro-fabricated structures.

U.S. Pat. No. 8,303,500 pertains to a wearable/handheld personal communication device with hardware and software sensor modules that sense and analyze all caregiver prescribed/monitored user-lifestyle activities, and deploys such analysis in improving user's overall health in terms of reduced risks for all-cause morbidities/mortalities and eventually a life without drugs. Termed Rx Zero, such method may be prescribed not just for maintaining a healthy lifestyle, but for treatment of chronic diseases with intent to wean the patients to minimal or zero pharmacological intervention, or in combination with medications to improve prognosis of the disease under treatment. The benefits of the Rx Zero method of the present invention extend not only to the individual and the community through high quality healthcare at lower cost, but payers by reducing the loss ratio on account of reduced cost of medical claims, and to the caregivers in terms of an effective tool that disseminates, implements and redefines “Primary Health Care” and “Prevention” at levels beyond the terms' currently understood scope that has transformed healthcare to sick care.

U.S. Reissued Pat. 38,749 pertains to a chronotherapy exercise technique for treating a patient whose abnormal condition, regardless of its nature or origin, is reflected by a heart rate variability (HRV) that is reduced and deviates from an HRV reflecting a normal condition. In this treatment, the patient in an exercise session undergoes a series of exercise-relaxation cycles in which during each cycle the pulse rate of the patient rises and then falls to generate a heart wave. To enhance the efficacy of the treatment, the heart waves generated in the course of an exercise session are synchronized in time with an internal wave produced by a biological clock, this activity functioning to expand the range of the biological wave and inducing the HRV to approach an HRV, which for the patient being treated reflects a normal condition.

U.S. Patent Application 2005/0015122 pertains to systems and methods for controllably adjusting the circadian pacemaker cycle of a subject using light (or other stimulus) through application of model-based predictive control techniques. This approach allows the use of closed-loop feedback to compensate for modeling errors, unknown initial conditions and disturbances. It also allows an optimal level of light (or other stimulus) to be generated based on minimization of a cost function. The cost function may incorporate a term associated with tracking errors and a term associated with the amount of light used. The tracking function may be minimized subject to one or more constraints which may include a minimum and maximum amount of light (or other stimulus).

Various systems and methodologies are known in the art. However, their structure and means of operation are substantially different from the present disclosure. The other inventions fail to solve all the problems taught by the present disclosure. The present invention and its embodiments take a holistic approach to correct and/or engage a patient's natural biorhythms with traditional medical treatments. At least one embodiment of this invention is presented in the drawings below and will be described in more detail herein.

### SUMMARY OF THE EMBODIMENTS

The present invention and its embodiments relate to treatments for various disease states involving fundamental modifications or tuning to at least one biological rhythm associated with a user. Disruption of biological rhythms, such as the menstrual cycle and other biologically programmed temporal processes, increases the risk and burden of disease. Via continuous actigraphy, a user can have their activities, environment, and physiological states measured. A software platform, such as a web based application or a mobile based application, can operate in conjunction with the actigraphy system. The timing and intensity of such variables can be algorithmically compared to baseline data based on a particular user or a particular demographic. Disruptions to the user's biological rhythms can then be identified and corrected as necessary.

The present invention and its embodiments provide a non-invasive, comprehensive, systematic approach to modifying these biological rhythms. Previous endeavors include point measurements and spot treatments including prescribing melatonin to increase drowsiness and phototherapy for seasonal affective disorder. Even yet, some application require invasive measures such as blood draws or transdermal delivery of drugs. Such methodologies are not desirable because they can cause undue stress on a user and do not allow for continual monitoring of the user. The present invention permits intelligent actions to be taken on quality data and data analyses.

In one embodiment there is a system for modifying at least one biological rhythm of an individual, the system having at least one sensor capable of measuring a physiological output of at least one user when kept in a proximity to the at least one user; a wireless transceiver that transmits data associated with the at least one sensor; a memory that stores the data associated with the at least one sensor; a control unit operably coupled to the at least one sensor, the wireless transceiver, and the memory, wherein the control unit enables the at least one user to interact with the system.

In another embodiment there is a system for modifying at least one biological rhythm of an individual, the system having at least one monitoring apparatus capable of monitoring at least one biorhythmic process, wherein the at least one monitoring apparatus has an accelerometer and an ambient light sensor; an electronic device capable of communicating with the at least one monitoring unit, wherein the electronic device has a processor, a memory, and instructions for executing at least one program contained thereon, wherein the at least one program analyzes data from the monitoring unit to calculate an index of circadian entrainment, wherein the index of circadian entrainment is used to present feedback to a user.

In another embodiment there is a system for modifying at least one biological rhythm of an individual, the system having monitoring apparatus comprising, at least one sensing module, the at least one sensing module being capable of sensing ambient light, acceleration, and/or gravitational forces, at least one power source operably coupled to the at least one sensing module, a display module capable of displaying data relating to at least the one sensing module and/or at least one power source, wherein the display module has a touch sensitive interface, a motor unit disposed within either the display module or any of the at least one sensing modules, and at least one light source; and electronic device capable of communicating with the at least one monitoring unit, wherein the electronic device has a processor, a memory, and instructions for executing at least one program contained thereon, wherein the at least one program analyzes data from the monitoring unit to calculate an index of circadian entrainment, wherein the index of circadian entrainment is used to present feedback to a user.

In another aspect of the present invention there is a method for tuning and/or modifying at least one biological rhythm of an individual, the method having the steps of at least one user having a monitoring apparatus located in a proximity to the at least one user; the monitoring apparatus collecting data via a least one sensor in the monitoring apparatus, wherein the data pertains to physiological and/or environmental variables related to the at least one user; analyzing the data; and the monitoring apparatus making at least one recommendation to the at least one user based on the analyzed data.

In another embodiment of the present invention there is a method for tuning and/or modifying at least one biological rhythm of an individual, the method having the steps of positioning a plurality of sensors to obtain one or more physiological and/or environmental parameters; collecting data via the plurality of sensors, wherein the plurality of sensors enables collection of data attributable to the one or more physiological and/or environmental parameters related to the at least one user; analyzing the data via a processor of the monitoring apparatus, wherein known values are compared to the one or more physiological and/or environmental parameters; and the monitoring apparatus making at least one recommendation to the at least one user based on the analyzed data.

In another aspect of the present invention there is a computer program embodied in a non-transitory computer-readable medium comprising computer readable instructions, which when executed by a processor, cause the processor to perform a method of instructing and/or causing modification to a biological rhythm of an individual, the method having the steps of collecting data from a plurality of sensors, wherein the data pertains to the physiological and/or environmental state in which the plurality of sensors is present; analyzing via a processor the data collected by the plurality of sensors; and the processor making at least one recommendation based on the performed analysis.

In another aspect of the present invention there is a monitoring apparatus having at least one sensing module, wherein the at least one sensing module is capable of sensing an environmental and/or physiological variable; at least one power source operably coupled to the at least one sensing module; a display module capable of displaying data relating to at least the one sensing module and/or at least one power source.

In yet another embodiment of the present invention there is a monitoring apparatus having at least one sensing module, the at least one sensing module being capable of sensing ambient light, acceleration, and/or gravitational forces; at least one power source operably coupled to the at least one sensing module; a display module capable of displaying data relating to at least the one sensing module and/or at least one power source; wherein the display module has a touch sensitive interface; a motor unit disposed within either the display module or any of the at least one sensing modules; and at least one light source.

In general, the present invention succeeds in conferring the following, and others not mentioned, benefits and objectives.

It is an object of the present invention to provide a system that is used to tune and/or modify at least one biological rhythm.

It is an object of the present invention to provide a system to prevent and/or alleviate disease.

It is an object of the present invention to provide a system to increase the quality of life of a user.

It is an object of the present invention to provide a system that measures physiological and environmental factors to calculate and remedy biological rhythm disruption.

It is an object of the present invention to provide a system that provides recommendations to a user to modify disruptive behaviors.

It is an object of the present invention to provide a system that uses a closed-loop feedback program.

It is an object of the present invention to provide a system that provides real time notifications and alerts.

It is an object of the present invention to provide a system that provides feedback to a caregiver or other third party.

It is an object of the present invention to provide a system that recommends when medications and/or medical procedures should be administered.

### BRIEF DESCRIPTION OF THE DRAWINGS

FIG. 1 is a high level overview of an embodiment of the present invention.

FIG. 2 is a perspective view of an embodiment of a monitoring apparatus in accordance with the present invention.

FIG. 3 is a flowchart detailing an overview method of use of a monitoring apparatus.

FIG. 4 is a flowchart detailing a method of setting up a monitoring apparatus.

FIG. 5 is an exemplary screen from an application selection screen in accordance with an embodiment of the present invention.

FIG. 6 is an exemplary screen from a questionnaire screen in accordance with an embodiment of the present invention.

FIG. 7 is an exemplary screen from a recommendation screen in accordance with an embodiment of the present invention.

### DESCRIPTION OF THE PREFERRED EMBODIMENTS

The preferred embodiments of the present invention will now be described with reference to the drawings. Identical elements in the various figures are identified with the same reference numerals.

Reference will now be made in detail to each embodiment of the present invention. Such embodiments are provided by way of explanation of the present invention, which is not intended to be limited thereto. In fact, those of ordinary skill in the art may appreciate upon reading the present specification and viewing the present drawings that various modifications and variations can be made thereto.

### Systems, Devices and Operating Systems

Typically, a user or users, which may be people or groups of users and/or other systems, may engage information technology systems (e.g., computers) to facilitate operation of the system and information processing. In turn, computers employ processors to process information and such processors may be referred to as central processing units (CPU). One form of processor is referred to as a microprocessor. CPUs use communicative circuits to pass binary encoded signals acting as instructions to enable various operations. These instructions may be operational and/or data instructions containing and/or referencing other instructions and data in various processor accessible and operable areas of memory (e.g., registers, cache memory, random access memory, etc.). Such communicative instructions may be stored and/or transmitted in batches (e.g., batches of instructions) as programs and/or data components to facilitate desired operations. These stored instruction codes, e.g., programs, may engage the CPU circuit components and other motherboard and/or system components to perform desired operations. One type of program is a computer operating system, which, may be executed by CPU on a computer; the operating system enables and facilitates users to access and operate computer information technology and resources. Some resources that may be employed in information technology systems include: input and output mechanisms through which data may pass into and out of a computer; memory storage into which data may be saved; and processors by which information may be processed. These information technology systems may be used to collect data for later retrieval, analysis, and manipulation, which may be facilitated through a database program. These information technology systems provide interfaces that allow users to access and operate various system components.

In one embodiment, the present invention may be connected to and/or communicate with entities such as, but not limited to: one or more users from user input devices; peripheral devices; an optional cryptographic processor device; and/or a communications network. For example, the present invention may be connected to and/or communicate with users, operating client device(s), including, but not limited to, personal computer(s), server(s) and/or various mobile device(s) including, but not limited to, cellular telephone(s), smartphone(s) (e.g., iPhone®, Blackberry®, Android OS-based phones etc.), tablet computer(s) (e.g., Apple iPad™, HP Slate™, Motorola Xoom™, etc.), eBook reader(s) (e.g., Amazon Kindle™, Barnes and Noble's Nook™ eReader, etc.), laptop computer(s), notebook(s), netbook(s), gaming console(s) (e.g., XBOX Live™, Nintendo® DS, Sony PlayStation® Portable, etc.), portable scanner(s) and/or the like.

Networks are commonly thought to comprise the interconnection and interoperation of clients, servers, and intermediary nodes in a graph topology. It should be noted that the term “server” as used throughout this application refers generally to a computer, other device, program, or combination thereof that processes and responds to the requests of remote users across a communications network. Servers serve their information to requesting “clients.” The term “client” as used herein refers generally to a computer, program, other device, user and/or combination thereof that is capable of processing and making requests and obtaining and processing any responses from servers across a communications network. A computer, other device, program, or combination thereof that facilitates, processes information and requests, and/or furthers the passage of information from a source user to a destination user is commonly referred to as a “node.” Networks are generally thought to facilitate the transfer of information from source points to destinations. A node specifically tasked with furthering the passage of information from a source to a destination is commonly called a “router.” There are many forms of networks such as Local Area Networks (LANs), Pico networks, Wide Area Networks (WANs), Wireless Networks (WLANs), etc. For example, the Internet is generally accepted as being an interconnection of a multitude of networks whereby remote clients and servers may access and interoperate with one another.

The present invention may be based on computer systems that may comprise, but are not limited to, components such as: a computer systemization connected to memory.

Computer Systemization

A computer systemization may comprise a clock, central processing unit (“CPU(s)” and/or “processor(s)” (these terms are used interchangeable throughout the disclosure unless noted to the contrary)), a memory (e.g., a read only memory (ROM), a random access memory (RAM), etc.), and/or an interface bus, and most frequently, although not necessarily, are all interconnected and/or communicating through a system bus on one or more (mother)board(s) having conductive and/or otherwise transportive circuit pathways through which instructions (e.g., binary encoded signals) may travel to effect communications, operations, storage, etc. Optionally, the computer systemization may be connected to an internal power source; e.g., optionally the power source may be internal. Optionally, a cryptographic processor and/or transceivers (e.g., ICs) may be connected to the system bus. In another embodiment, the cryptographic processor and/or transceivers may be connected as either internal and/or external peripheral devices via the interface bus I/O. In turn, the transceivers may be connected to antenna(s), thereby effectuating wireless transmission and reception of various communication and/or sensor protocols; for example the antenna(s) may connect to: a Texas Instruments WiLink WL1283 transceiver chip (e.g., providing 802.11n, Bluetooth 3.0, FM, global positioning system (GPS) (thereby allowing the controller of the present invention to determine its location)); Broadcom BCM4329FKUBG transceiver chip (e.g., providing 802.11n, Bluetooth 2.1+EDR, FM, etc.); a Broadcom BCM4750IUB8 receiver chip (e.g., GPS); an Infineon Technologies X-Gold 618-PMB9800 (e.g., providing 2G/3G HSDPA/HSUPA communications); and/or the like. The system clock typically has a crystal oscillator and generates a base signal through the computer systemization's circuit pathways. The clock is typically coupled to the system bus and various clock multipliers that will increase or decrease the base operating frequency for other components interconnected in the computer systemization. The clock and various components in a computer systemization drive signals embodying information throughout the system. Such transmission and reception of instructions embodying information throughout a computer systemization may be commonly referred to as communications. These communicative instructions may further be transmitted, received, and the cause of return and/or reply communications beyond the instant computer systemization to: communications networks, input devices, other computer systemizations, peripheral devices, and/or the like. Of course, any of the above components may be connected directly to one another, connected to the CPU, and/or organized in numerous variations employed as exemplified by various computer systems.

The CPU comprises at least one high-speed data processor adequate to execute program components for executing user and/or system-generated requests. Often, the processors themselves will incorporate various specialized processing units, such as, but not limited to: integrated system (bus) controllers, memory management control units, floating point units, and even specialized processing sub-units like graphics processing units, digital signal processing units, and/or the like. Additionally, processors may include internal fast access addressable memory, and be capable of mapping and addressing memory beyond the processor itself; internal memory may include, but is not limited to: fast registers, various levels of cache memory (e.g., level 1, 2, 3, etc.), RAM, etc. The processor may access this memory through the use of a memory address space that is accessible via instruction address, which the processor can construct and decode allowing it to access a circuit path to a specific memory address space having a memory state. The CPU may be a microprocessor such as: AMD's Athlon, Duron and/or Opteron; ARM's application, embedded and secure processors; IBM and/or Motorola's DragonBall and PowerPC; IBM's and Sony's Cell processor; Intel's Celeron, Core (2) Duo, Itanium, Pentium, Xeon, and/or XScale; and/or the like processor(s). The CPU interacts with memory through instruction passing through conductive and/or transportive conduits (e.g., (printed) electronic and/or optic circuits) to execute stored instructions (i.e., program code) according to conventional data processing techniques. Such instruction passing facilitates communication within the present invention and beyond through various interfaces. Should processing requirements dictate a greater amount speed and/or capacity, distributed processors (e.g., Distributed embodiments of the present invention), mainframe, multi-core, parallel, and/or super-computer architectures may similarly be employed. Alternatively, should deployment requirements dictate greater portability, smaller Personal Digital Assistants (PDAs) may be employed.

Depending on the particular implementation, features of the present invention may be achieved by implementing a microcontroller such as CAST's R8051XC2 microcontroller; Intel's MCS 51 (i.e., 8051 microcontroller); and/or the like. Also, to implement certain features of the various embodiments, some feature implementations may rely on embedded components, such as: Application-Specific Integrated Circuit (“ASIC”), Digital Signal Processing (“DSP”), Field Programmable Gate Array (“FPGA”), and/or the like embedded technology. For example, any of the component collection (distributed or otherwise) and/or features of the present invention may be implemented via the microprocessor and/or via embedded components; e.g., via ASIC, coprocessor, DSP, FPGA, and/or the like. Alternately, some implementations of the present invention may be implemented with embedded components that are configured and used to achieve a variety of features or signal processing.

Depending on the particular implementation, the embedded components may include software solutions, hardware solutions, and/or some combination of both hardware/software solutions. For example, features of the present invention discussed herein may be achieved through implementing FPGAs, which are a semiconductor devices containing programmable logic components called “logic blocks”, and programmable interconnects, such as the high performance FPGA Virtex series and/or the low cost Spartan series manufactured by Xilinx. Logic blocks and interconnects can be programmed by the customer or designer, after the FPGA is manufactured, to implement any of the features of the present invention. A hierarchy of programmable interconnects allow logic blocks to be interconnected as needed by the system designer/administrator of the present invention, somewhat like a one-chip programmable breadboard. An FPGA's logic blocks can be programmed to perform the function of basic logic gates such as AND, and XOR, or more complex combinational functions such as decoders or simple mathematical functions. In most FPGAs, the logic blocks also include memory elements, which may be simple flip-flops or more complete blocks of memory. In some circumstances, the present invention may be developed on regular FPGAs and then migrated into a fixed version that more resembles ASIC implementations. Alternate or coordinating implementations may migrate features of the controller of the present invention to a final ASIC instead of or in addition to FPGAs. Depending on the implementation all of the aforementioned embedded components and microprocessors may be considered the “CPU” and/or “processor” for the present invention.

Power Source

The power source may be of any standard form for powering small electronic circuit board devices such as the following power cells: alkaline, lithium hydride, lithium ion, lithium polymer, nickel cadmium, solar cells, and/or the like. Other types of AC or DC power sources may be used as well. In the case of solar cells, in one embodiment, the case provides an aperture through which the solar cell may capture photonic energy. The power cell is connected to at least one of the interconnected subsequent components of the present invention thereby providing an electric current to all subsequent components. In one example, the power source is connected to the system bus component. In an alternative embodiment, an outside power source is provided through a connection across the I/O interface. For example, a USB and/or IEEE 1394 connection carries both data and power across the connection and is therefore a suitable source of power.

Interface Adapters

Interface bus(ses) may accept, connect, and/or communicate to a number of interface adapters, conventionally although not necessarily in the form of adapter cards, such as but not limited to: input output interfaces (I/O), storage interfaces, network interfaces, and/or the like. Optionally, cryptographic processor interfaces similarly may be connected to the interface bus. The interface bus provides for the communications of interface adapters with one another as well as with other components of the computer systemization. Interface adapters are adapted for a compatible interface bus. Interface adapters conventionally connect to the interface bus via a slot architecture. Conventional slot architectures may be employed, such as, but not limited to: Accelerated Graphics Port (AGP), Card Bus, (Extended) Industry Standard Architecture ((E)ISA), Micro Channel Architecture (MCA), NuBus, Peripheral Component Interconnect (Extended) (PCI(X)), PCI Express, Personal Computer Memory Card International Association (PCMCIA), and/or the like.

Storage interfaces may accept, communicate, and/or connect to a number of storage devices such as, but not limited to: storage devices, removable disc devices, and/or the like. Storage interfaces may employ connection protocols such as, but not limited to: (Ultra) (Serial) Advanced Technology Attachment (Packet Interface) ((Ultra) (Serial) ATA(PI)), (Enhanced) Integrated Drive Electronics ((E)IDE), Institute of Electrical and Electronics Engineers (IEEE) 1394, fiber channel, Small Computer Systems Interface (SCSI), Universal Serial Bus (USB), and/or the like.

Network interfaces may accept, communicate, and/or connect to a communications network. Through a communications network, the controller of the present invention is accessible through remote clients (e.g., computers with web browsers) by users. Network interfaces may employ connection protocols such as, but not limited to: direct connect, Ethernet (thick, thin, twisted pair 10/100/1000 Base T, and/or the like), Token Ring, wireless connection such as IEEE 802.11a-x, and/or the like. Should processing requirements dictate a greater amount speed and/or capacity, distributed network controllers (e.g., Distributed embodiments of the present invention), architectures may similarly be employed to pool, load balance, and/or otherwise increase the communicative bandwidth required by the controller of the present invention. A communications network may be any one and/or the combination of the following: a direct interconnection; the Internet; a Local Area Network (LAN); a Metropolitan Area Network (MAN); an Operating Missions as Nodes on the Internet (OMNI); a secured custom connection; a Wide Area Network (WAN); a wireless network (e.g., employing protocols such as, but not limited to a Wireless Application Protocol (WAP), I-mode, and/or the like); and/or the like. A network interface may be regarded as a specialized form of an input output interface. Further, multiple network interfaces may be used to engage with various communications network types. For example, multiple network interfaces may be employed to allow for the communication over broadcast, multicast, and/or unicast networks.

Input Output interfaces (I/O) may accept, communicate, and/or connect to user input devices, peripheral devices, cryptographic processor devices, and/or the like. I/O may employ connection protocols such as, but not limited to: audio: analog, digital, monaural, RCA, stereo, and/or the like; data: Apple Desktop Bus (ADB), IEEE 1394a-b, serial, universal serial bus (USB); infrared; joystick; keyboard; midi; optical; PC AT; PS/2; parallel; radio; video interface: Apple Desktop Connector (ADC), BNC, coaxial, component, composite, digital, Digital Visual Interface (DVI), high-definition multimedia interface (HDMI), RCA, RF antennae, S-Video, VGA, and/or the like; wireless transceivers: 802.11a/b/g/n/x; Bluetooth; cellular (e.g., code division multiple access (CDMA), high speed packet access (HSPA(+)), high-speed downlink packet access (HSDPA), global system for mobile communications (GSM), long term evolution (LTE), WiMax, etc.); and/or the like. One typical output device may include a video display, which typically comprises a Cathode Ray Tube (CRT) or Liquid Crystal Display (LCD) based monitor with an interface (e.g., DVI circuitry and cable) that accepts signals from a video interface, may be used. The video interface composites information generated by a computer systemization and generates video signals based on the composited information in a video memory frame. Another output device is a television set, which accepts signals from a video interface. Typically, the video interface provides the composited video information through a video connection interface that accepts a video display interface (e.g., an RCA composite video connector accepting an RCA composite video cable; a DVI connector accepting a DVI display cable, etc.).

User input devices often are a type of peripheral device (see below) and may include: card readers, dongles, finger print readers, gloves, graphics tablets, joysticks, keyboards, microphones, mouse (mice), remote controls, retina readers, touch screens (e.g., capacitive, resistive, etc.), trackballs, trackpads, sensors (e.g., accelerometers, ambient light, GPS, gyroscopes, proximity, etc.), styluses, and/or the like.

Peripheral devices and the like may be connected and/or communicate to I/O and/or other facilities of the like such as network interfaces, storage interfaces, directly to the interface bus, system bus, the CPU, and/or the like. Peripheral devices may be external, internal and/or part of the controller of the present invention. Peripheral devices may also include, for example, an antenna, audio devices (e.g., line-in, line-out, microphone input, speakers, etc.), cameras (e.g., still, video, webcam, etc.), drive motors, lighting, video monitors and/or the like.

Cryptographic units such as, but not limited to, microcontrollers, processors, interfaces, and/or devices may be attached, and/or communicate with the controller of the present invention. A MC68HC16 microcontroller, manufactured by Motorola Inc., may be used for and/or within cryptographic units. The MC68HC16 microcontroller utilizes a 16-bit multiply-and-accumulate instruction in the 16 MHz configuration and requires less than one second to perform a 512-bit RSA private key operation. Cryptographic units support the authentication of communications from interacting agents, as well as allowing for anonymous transactions. Cryptographic units may also be configured as part of CPU. Equivalent microcontrollers and/or processors may also be used. Other commercially available specialized cryptographic processors include: the Broadcom's CryptoNetX and other Security Processors; nCipher's nShield, SafeNet's Luna PCI (e.g., 7100) series; Semaphore Communications' 40 MHz Roadrunner 184; Sun's Cryptographic Accelerators (e.g., Accelerator 6000 PCIe Board, Accelerator 500 Daughtercard); Via Nano Processor (e.g., L2100, L2200, U2400) line, which is capable of performing 500+MB/s of cryptographic instructions; VLSI Technology's 33 MHz 6868; and/or the like.

Memory

Generally, any mechanization and/or embodiment allowing a processor to affect the storage and/or retrieval of information is regarded as memory. However, memory is a fungible technology and resource, thus, any number of memory embodiments may be employed in lieu of or in concert with one another. It is to be understood that the controller of the present invention and/or a computer systemization may employ various forms of memory. For example, a computer systemization may be configured wherein the functionality of on-chip CPU memory (e.g., registers), RAM, ROM, and any other storage devices are provided by a paper punch tape or paper punch card mechanism; of course such an embodiment would result in an extremely slow rate of operation. In a typical configuration, memory will include ROM, RAM, and a storage device. A storage device may be any conventional computer system storage. Storage devices may include a drum; a (fixed and/or removable) magnetic disk drive; a magneto-optical drive; an optical drive (i.e., Blueray, CD ROM/RAM/Recordable (R)/ReWritable (RW), DVD R/RW, HD DVD R/RW etc.); an array of devices (e.g., Redundant Array of Independent Disks (RAID)); solid state memory devices (USB memory, solid state drives (SSD), etc.); other processor-readable storage mediums; and/or other devices of the like. Thus, a computer systemization generally requires and makes use of memory.

Component Collection

The memory may contain a collection of program and/or database components and/or data such as, but not limited to: operating system component(s) (operating system); information server component(s) (information server); user interface component(s) (user interface); Web browser component(s) (Web browser); database(s); mail server component(s); mail client component(s); cryptographic server component(s) (cryptographic server) and/or the like (i.e., collectively a component collection). These components may be stored and accessed from the storage devices and/or from storage devices accessible through an interface bus. Although non-conventional program components such as those in the component collection, typically, are stored in a local storage device, they may also be loaded and/or stored in memory such as: peripheral devices, RAM, remote storage facilities through a communications network, ROM, various forms of memory, and/or the like.

Operating System

The operating system component is an executable program component facilitating the operation of the controller of the present invention. Typically, the operating system facilitates access of I/O, network interfaces, peripheral devices, storage devices, and/or the like. The operating system may be a highly fault tolerant, scalable, and secure system such as: Apple Macintosh OS X (Server); AT&T Plan 9; Be OS; Unix and Unix-like system distributions (such as AT&T's UNIX; Berkley Software Distribution (BSD) variations such as FreeBSD, NetBSD, OpenBSD, and/or the like; Linux distributions such as Red Hat, Ubuntu, and/or the like); and/or the like operating systems. However, more limited and/or less secure operating systems also may be employed such as Apple Macintosh OS, IBM OS/2, Microsoft DOS, Microsoft Windows 2000/2003/3.1/95/98/CE/Millennium/NT/Vista/XP (Server), Palm OS, and/or the like. The operating system may be one specifically optimized to be run on a mobile computing device, such as iOS, Android, Windows Phone, Tizen, Symbian, and/or the like. An operating system may communicate to and/or with other components in a component collection, including itself, and/or the like. Most frequently, the operating system communicates with other program components, user interfaces, and/or the like. For example, the operating system may contain, communicate, generate, obtain, and/or provide program component, system, user, and/or data communications, requests, and/or responses. The operating system, once executed by the CPU, may enable the interaction with communications networks, data, I/O, peripheral devices, program components, memory, user input devices, and/or the like. The operating system may provide communications protocols that allow the controller of the present invention to communicate with other entities through a communications network. Various communication protocols may be used by the controller of the present invention as a subcarrier transport mechanism for interaction, such as, but not limited to: multicast, TCP/IP, UDP, unicast, and/or the like.

Information Server

An information server component is a stored program component that is executed by a CPU. The information server may be a conventional Internet information server such as, but not limited to Apache Software Foundation's Apache, Microsoft's Internet Information Server, and/or the like. The information server may allow for the execution of program components through facilities such as Active Server Page (ASP), ActiveX, (ANSI) (Objective−) C (++), C# and/or .NET, Common Gateway Interface (CGI) scripts, dynamic (D) hypertext markup language (HTML), FLASH, Java, JavaScript, Practical Extraction Report Language (PERL), Hypertext Pre-Processor (PHP), pipes, Python, wireless application protocol (WAP), WebObjects, and/or the like. The information server may support secure communications protocols such as, but not limited to, File Transfer Protocol (FTP); HyperText Transfer Protocol (HTTP); Secure Hypertext Transfer Protocol (HTTPS), Secure Socket Layer (SSL), messaging protocols (e.g., America Online (AOL) Instant Messenger (AIM), Application Exchange (APEX), ICQ, Internet Relay Chat (IRC), Microsoft Network (MSN) Messenger Service, Presence and Instant Messaging Protocol (PRIM), Internet Engineering Task Force's (IETF's) Session Initiation Protocol (SIP), SIP for Instant Messaging and Presence Leveraging Extensions (SIMPLE), open XML-based Extensible Messaging and Presence Protocol (XMPP) (i.e., Jabber or Open Mobile Alliance's (OMA's) Instant Messaging and Presence Service (IMPS)), Yahoo! Instant Messenger Service, and/or the like. The information server provides results in the form of Web pages to Web browsers, and allows for the manipulated generation of the Web pages through interaction with other program components. After a Domain Name System (DNS) resolution portion of an HTTP request is resolved to a particular information server, the information server resolves requests for information at specified locations on the controller of the present invention based on the remainder of the HTTP request. For example, a request such as http://123.124.125.126/myInformation.html might have the IP portion of the request “123.124.125.126” resolved by a DNS server to an information server at that IP address; that information server might in turn further parse the http request for the “/myInformation.html” portion of the request and resolve it to a location in memory containing the information “myInformation.html.” Additionally, other information serving protocols may be employed across various ports, e.g., FTP communications across port, and/or the like. An information server may communicate to and/or with other components in a component collection, including itself, and/or facilities of the like. Most frequently, the information server communicates with the database of the present invention, operating systems, other program components, user interfaces, Web browsers, and/or the like.

Access to the database of the present invention may be achieved through a number of database bridge mechanisms such as through scripting languages as enumerated below (e.g., CGI) and through inter-application communication channels as enumerated below (e.g., CORBA, WebObjects, etc.). Any data requests through a Web browser are parsed through the bridge mechanism into appropriate grammars as required by the present invention. In one embodiment, the information server would provide a Web form accessible by a Web browser. Entries made into supplied fields in the Web form are tagged as having been entered into the particular fields, and parsed as such. The entered terms are then passed along with the field tags, which act to instruct the parser to generate queries directed to appropriate tables and/or fields. In one embodiment, the parser may generate queries in standard SQL by instantiating a search string with the proper join/select commands based on the tagged text entries, wherein the resulting command is provided over the bridge mechanism to the present invention as a query. Upon generating query results from the query, the results are passed over the bridge mechanism, and may be parsed for formatting and generation of a new results Web page by the bridge mechanism. Such a new results Web page is then provided to the information server, which may supply it to the requesting Web browser.

Also, an information server may contain, communicate, generate, obtain, and/or provide program component, system, user, and/or data communications, requests, and/or responses.

User Interface

Computer interfaces in some respects are similar to automobile operation interfaces. Automobile operation interface elements such as steering wheels, gearshifts, and speedometers facilitate the access, operation, and display of automobile resources, and status. Computer interaction interface elements such as check boxes, cursors, menus, scrollers, and windows (collectively and commonly referred to as widgets) similarly facilitate the access, capabilities, operation, and display of data and computer hardware and operating system resources, and status. Operation interfaces are commonly called user interfaces. Graphical user interfaces (GUIs) such as the Apple Macintosh Operating System's Aqua, IBM's OS/2, Microsoft's Windows 2000/2003/3.1/95/98/CE/Millennium/NT/XP/Vista/7 (i.e., Aero), Unix's X-Windows (e.g., which may include additional Unix graphic interface libraries and layers such as K Desktop Environment (KDE), mythTV and GNU Network Object Model Environment (GNOME)), web interface libraries (e.g., ActiveX, AJAX, (D)HTML, FLASH, Java, JavaScript, etc. interface libraries such as, but not limited to, Dojo, jQuery(UI), MooTools, Prototype, script.aculo.us, SWFObject, Yahoo! User Interface, any of which may be used and) provide a baseline and means of accessing and displaying information graphically to users.

A user interface component is a stored program component that is executed by a CPU. The user interface may be a conventional graphic user interface as provided by, with, and/or atop operating systems and/or operating environments such as already discussed. The user interface may allow for the display, execution, interaction, manipulation, and/or operation of program components and/or system facilities through textual and/or graphical facilities. The user interface provides a facility through which users may affect, interact, and/or operate a computer system. A user interface may communicate to and/or with other components in a component collection, including itself, and/or facilities of the like. Most frequently, the user interface communicates with operating systems, other program components, and/or the like. The user interface may contain, communicate, generate, obtain, and/or provide program component, system, user, and/or data communications, requests, and/or responses.

Web Browser

A Web browser component is a stored program component that is executed by a CPU. The Web browser may be a conventional hypertext viewing application such as Microsoft Internet Explorer or Netscape Navigator. Secure Web browsing may be supplied with 128 bit (or greater) encryption by way of HTTPS, SSL, and/or the like. Web browsers allowing for the execution of program components through facilities such as ActiveX, AJAX, (D)HTML, FLASH, Java, JavaScript, web browser plug-in APIs (e.g., FireFox, Safari Plug-in, and/or the like APIs), and/or the like. Web browsers and like information access tools may be integrated into PDAs, cellular telephones, and/or other mobile devices. A Web browser may communicate to and/or with other components in a component collection, including itself, and/or facilities of the like. Most frequently, the Web browser communicates with information servers, operating systems, integrated program components (e.g., plug-ins), and/or the like; e.g., it may contain, communicate, generate, obtain, and/or provide program component, system, user, and/or data communications, requests, and/or responses. Of course, in place of a Web browser and information server, a combined application may be developed to perform similar functions of both. The combined application would similarly affect the obtaining and the provision of information to users, user agents, and/or the like from the enabled nodes of the present invention. The combined application may be nugatory on systems employing standard Web browsers.

Mail Server

A mail server component is a stored program component that is executed by a CPU. The mail server may be a conventional Internet mail server such as, but not limited to sendmail, Microsoft Exchange, and/or the like. The mail server may allow for the execution of program components through facilities such as ASP, ActiveX, (ANSI) (Objective−) C (++), C# and/or .NET, CGI scripts, Java, JavaScript, PERL, PHP, pipes, Python, WebObjects, and/or the like. The mail server may support communications protocols such as, but not limited to: Internet message access protocol (IMAP), Messaging Application Programming Interface (MAPI)/Microsoft Exchange, post office protocol (POP3), simple mail transfer protocol (SMTP), and/or the like. The mail server can route, forward, and process incoming and outgoing mail messages that have been sent, relayed and/or otherwise traversing through and/or to the present invention.

Access to the mail of the present invention may be achieved through a number of APIs offered by the individual Web server components and/or the operating system.

Also, a mail server may contain, communicate, generate, obtain, and/or provide program component, system, user, and/or data communications, requests, information, and/or responses.

Mail Client

A mail client component is a stored program component that is executed by a CPU. The mail client may be a conventional mail viewing application such as Apple Mail, Microsoft Entourage, Microsoft Outlook, Microsoft Outlook Express, Mozilla, Thunderbird, and/or the like. Mail clients may support a number of transfer protocols, such as: IMAP, Microsoft Exchange, POP3, SMTP, and/or the like. A mail client may communicate to and/or with other components in a component collection, including itself, and/or facilities of the like. Most frequently, the mail client communicates with mail servers, operating systems, other mail clients, and/or the like; e.g., it may contain, communicate, generate, obtain, and/or provide program component, system, user, and/or data communications, requests, information, and/or responses. Generally, the mail client provides a facility to compose and transmit electronic mail messages.

Cryptographic Server

A cryptographic server component is a stored program component that is executed by a CPU, cryptographic processor, cryptographic processor interface, cryptographic processor device, and/or the like. Cryptographic processor interfaces will allow for expedition of encryption and/or decryption requests by the cryptographic component; however, the cryptographic component, alternatively, may run on a conventional CPU. The cryptographic component allows for the encryption and/or decryption of provided data. The cryptographic component allows for both symmetric and asymmetric (e.g., Pretty Good Protection (PGP)) encryption and/or decryption. The cryptographic component may employ cryptographic techniques such as, but not limited to: digital certificates (e.g., X.509 authentication framework), digital signatures, dual signatures, enveloping, password access protection, public key management, and/or the like. The cryptographic component will facilitate numerous (encryption and/or decryption) security protocols such as, but not limited to: checksum, Data Encryption Standard (DES), Elliptical Curve Encryption (ECC), International Data Encryption Algorithm (IDEA), Message Digest 5 (MD5, which is a one way hash function), passwords, Rivest Cipher (RC5), Rijndael, RSA (which is an Internet encryption and authentication system that uses an algorithm developed in 1977 by Ron Rivest, Adi Shamir, and Leonard Adleman), Secure Hash Algorithm (SHA), Secure Socket Layer (SSL), Secure Hypertext Transfer Protocol (HTTPS), and/or the like. Employing such encryption security protocols, the present invention may encrypt all incoming and/or outgoing communications and may serve as node within a virtual private network (VPN) with a wider communications network. The cryptographic component facilitates the process of “security authorization” whereby access to a resource is inhibited by a security protocol wherein the cryptographic component effects authorized access to the secured resource. In addition, the cryptographic component may provide unique identifiers of content, e.g., employing and MD5 hash to obtain a unique signature for an digital audio file. A cryptographic component may communicate to and/or with other components in a component collection, including itself, and/or facilities of the like. The cryptographic component supports encryption schemes allowing for the secure transmission of information across a communications network to enable the component of the present invention to engage in secure transactions if so desired. The cryptographic component facilitates the secure accessing of resources on the present invention and facilitates the access of secured resources on remote systems; i.e., it may act as a client and/or server of secured resources. Most frequently, the cryptographic component communicates with information servers, operating systems, other program components, and/or the like. The cryptographic component may contain, communicate, generate, obtain, and/or provide program component, system, user, and/or data communications, requests, and/or responses.

A Database of the Present Invention

The database component of the present invention may be embodied in a database and its stored data. The database is a stored program component, which is executed by the CPU; the stored program component portion configuring the CPU to process the stored data. The database may be a conventional, fault tolerant, relational, scalable, secure database such as Oracle or Sybase. Relational databases are an extension of a flat file. Relational databases consist of a series of related tables. The tables are interconnected via a key field. Use of the key field allows the combination of the tables by indexing against the key field; i.e., the key fields act as dimensional pivot points for combining information from various tables. Relationships generally identify links maintained between tables by matching primary keys. Primary keys represent fields that uniquely identify the rows of a table in a relational database. More precisely, they uniquely identify rows of a table on the “one” side of a one-to-many relationship.

Alternatively, the database of the present invention may be implemented using various standard data-structures, such as an array, hash, (linked) list, struct, structured text file (e.g., XML), table, and/or the like. Such data-structures may be stored in memory and/or in (structured) files. In another alternative, an object-oriented database may be used, such as Frontier, ObjectStore, Poet, Zope, and/or the like. Object databases can include a number of object collections that are grouped and/or linked together by common attributes; they may be related to other object collections by some common attributes. Object-oriented databases perform similarly to relational databases with the exception that objects are not just pieces of data but may have other types of functionality encapsulated within a given object. If the database of the present invention is implemented as a data-structure, the use of the database of the present invention may be integrated into another component such as the component of the present invention. Also, the database may be implemented as a mix of data structures, objects, and relational structures. Databases may be consolidated and/or distributed in countless variations through standard data processing techniques. Portions of databases, e.g., tables, may be exported and/or imported and thus decentralized and/or integrated.

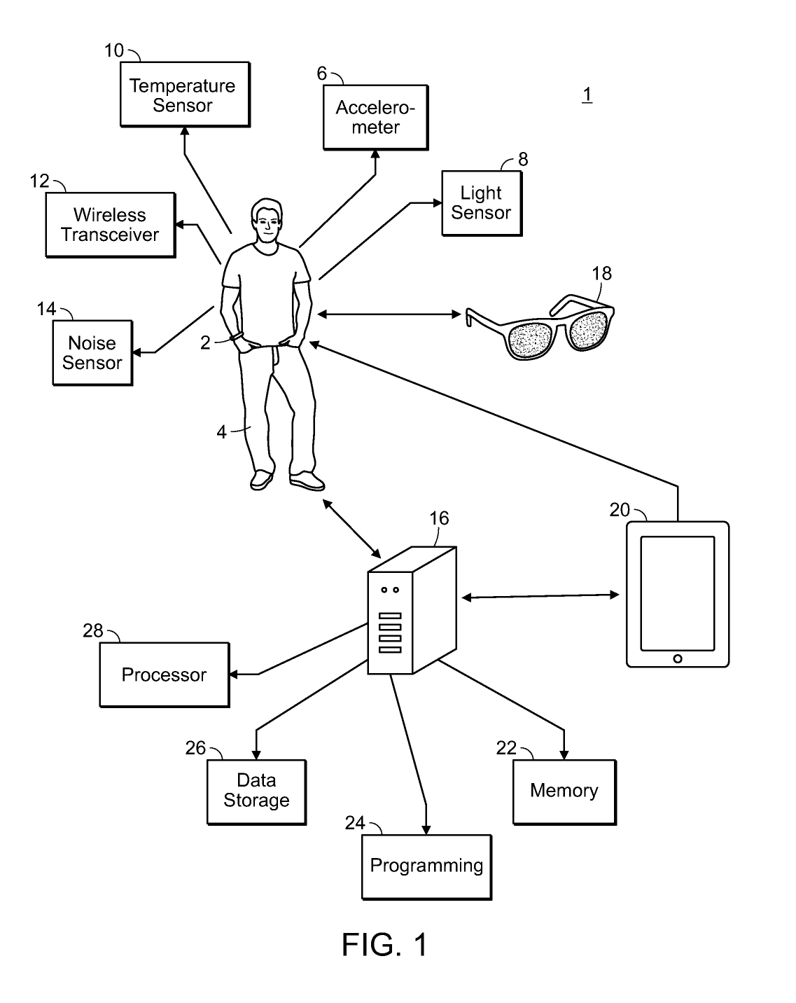

Referring now to FIG. 1, there is a high level overview of an embodiment of the present invention. The system 1 generally comprises a monitoring apparatus 2, an electronic device 20, light attenuating eyewear 18, and a server 16.

The monitoring apparatus 2 is intended to be worn by a mammal, preferably a human user 4, and may be worn around the human user's 4 wrist, neck, arm, leg, torso, or the like or some combination thereof. Generally, the monitoring apparatus 2 has a number of sensors including but not limited to a temperature sensor 10, light sensor 8, accelerometer 6, and noise sensor 14 or a combination thereof.

The temperature sensor 10 may be capable of taking both an ambient air temperature and a body temperature of a user or one of the other. In some instances, the temperature sensor 10 may only record one of such values and multiple temperature sensors may be required. The accelerometer 6, and in some instances digital gyroscope, provide movement and spatial orientation of the monitoring apparatus 2 as a user is both awake and asleep. The light sensor 8 preferably collects ambient light from the environment, and the noise sensor collects sounds from the ambient environment. All of such data may be collected and sent in real time via the wireless transceiver 12 enabling real time notifications in response to deviations or alignments with one's preferential rhythm. In some instances, the wireless transceiver 12 may be programmed to send collected data to the server 16 or electronic device 20 for storage and analysis at certain times or time periods.

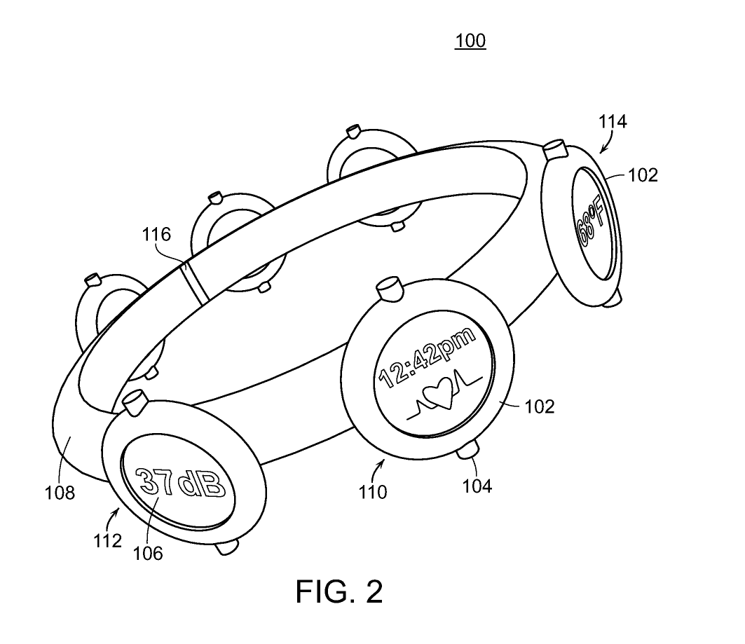

The monitoring apparatus 2 may take a number of forms, for example see FIG. 2, and may be customizable for or by the user. Further, a wireless transceiver 12 may be implemented to enable the sending and receiving of data wirelessly. Such data may be sent to the electronic device 20, the server 16, or other suitable electronic apparatus. A power source (not shown) such as a battery may also be implemented to provide electric power to the monitoring apparatus.

The light attenuating eyewear 18 may be sunglasses, glasses, or other eyewear that has lenses configured to attenuate certain wavelengths or ranges of wavelengths of light thereby promoting a natural circadian rhythm for the human user 4. In some embodiments the light attenuating eyewear 18 filters or blocks wavelengths of light ranging from about 300 nm to about 700 nm from reaching the retinas of the human user 4. More preferably, light ranging from about 400 nm to about 500 nm is attenuated and thereby limited or prevented from each the retinas of the human user 4. The light attenuating eyewear 18 may be placed upon the human user's 4 face upon a prescribed time frame or upon a recommendation provided by the monitoring apparatus 2 or electronic device 20. In some embodiments, an optional light box may be required to provide the user with bright light of a particular wavelength when no such light may be readily or practically available to the user.

The electronic device 20 may be any number of electronic devices capable of being connected to a communication network. The communication network may be a network such as the Internet or an intranet connection. In some instances, the electronic device 20 may communicate with the monitoring apparatus 2 or server 16 or a combination thereof using a dedicated communication network. In practice the electronic device 20 may be, but is not limited to, a smart phone, smart watch, laptop computer, desktop computer, tablet, PDA, gaming system, multimedia player, smart television, and the like or some combination thereof.

The server 16 preferably provides for at least a processor 28, data storage 26, programming 24, and a memory 22 and varying combinations thereof. The server 16 is preferably capable of communicating with the monitoring apparatus 2, the electronic device 20, or some combination thereof. Such communication enables the server 16 to store information collected or input into the monitoring apparatus 2 or electronic device 20. Further, the server 16 may store, as noted, programming which would enable the download and/or update of applications and programming running on either or both of the monitoring apparatus 2 and the electronic device 20.

The server 16 may be comprised of any number of servers capable of the above and other not mentioned capabilities. The servers may be in one location or positioned in remote locations in order to provide comprehensive transmission and collection and storage of data. In some instances, the data transmission and storage may enable the collected data to be shared with a third party such as a health care professional who may be able to “coach” or otherwise provide recommendations to the user.

Overall, the system provides, at least in part, a closed-loop feedback for cognitive behavioral therapy including modifications of biological rhythms including a user's circadian rhythm. The monitoring apparatus 2 provides for actigraphically measured and algorithmically analyzed results. The results may be analyzed in accordance with a variety or a combination of measures including sinusoidal curves, R² values, lunar/solar cycles, personal information, and the like. Some priority or hierarchy may be given to certain parameters or targets such as light blockage/therapy, sleep hygiene, meal timing, exercise timing, and medication timing. In some embodiments, priority may be given within those categories, and other categories not named, in order to provide a comprehensive analysis to the user. For example, a sleep medication/aid with a short acting soporific effect may be recommended over a sleep medication/aid with a long such effect.

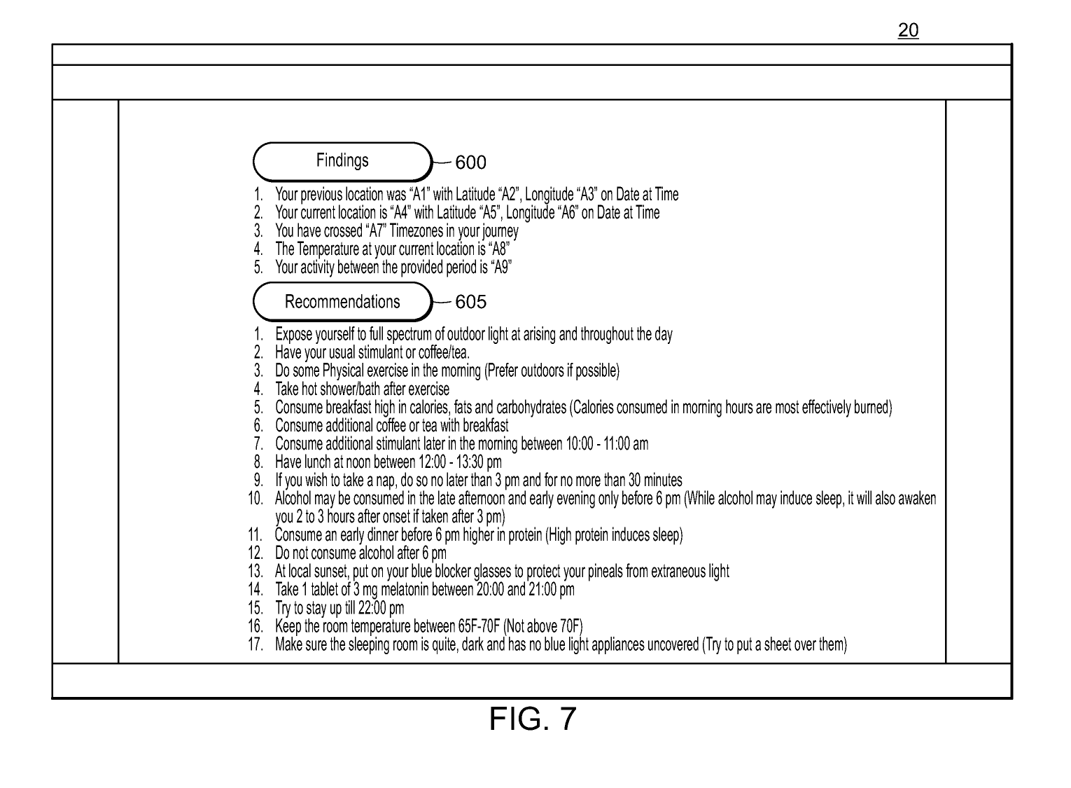

Such results may be accessible to the user or may simply be reported as recommendations for the user in order to more properly align or tune their biological rhythms (see FIG. 7). In some instances, a user may be able to access via the monitoring device 2 or the electronic device 20 a visual representation of an “ideal” rhythm with an overlay of their “actual” rhythm. Various factors may be able to show the amount of correction one can obtain be taking certain actions recommended by the system.

In addition to making recommendations, the system may provide the user, via the monitoring apparatus 2 and/or electronic device 20, with educational videos based on the analysis provided to the user. The educational videos may be designed to help a user properly understand and interpret the results, as well as ensure they are taking the proper corrective measures. These videos may be pushed to the user or may be available to the user via links or notifications.

FIG. 2 illustrates a perspective view of one embodiment of the monitoring apparatus 2 of the present invention. The monitoring apparatus 100, in general, preferably functions as an actigraphic tool that interfaces with the system as described in FIG. 1. Here, the various sensors implemented are modules 102 coupled to a band 108. The modules 102 may be readily removable and may be capable of being placed in varying positions around the band 108. Exemplary modules may include those as previously listed above and further include a heart rate module 110, a gyroscope module (not shown), and a bioimpedance module (not shown) and varying combinations thereof. As shown, the monitoring apparatus 2, in this embodiment, also includes a noise module 112, and a temperature module 114.

Each or any of the modules may include a display 106, vibrating motor (not shown), and at least one depressible or touch sensitive button 104. The display 106 may be a color or monochrome or black and white display capable of displaying numerals, characters, letters, symbols, and the like or some combination thereof. In some embodiments, the display 106 may be touch sensitive. Preferably, the display 106 is a liquid crystal display or electronic ink display. However, other displays may be implemented and differing modules may have differing display types.

The modules 102 in some embodiments may have covers that flip, screw or otherwise selectively cover the display 106 to prevent damage to the modules 102 during activity or when a user is sleeping. The modules 102 may snap into openings on the band 108 or may twist into position. Further, in some embodiments, there may be clasps or coupling mechanisms attached directly to the modules 102 with segments of band coupled thereto enabling the size of the band to be changed with the amount of modules 102 added to the configuration.

The touch sensitive button 104 may be disposed on varying surfaces (front, back, side, etc.) of each module on which a button is present. The touch sensitive button 104 may provide for varying functionality such as menu scrolling and selection, start/stop of timing, collection of data, transmission of data, turning on/off of LED or other display lighting, and the like or varying combinations thereof. In some embodiments the lighting is a RGB LED, whereas in other embodiments it is a phosphor-converted LED. Yet, in other embodiments, other suitable light sources may be employed.

Further, in some embodiments, the band 108 is capable of stretching and having resilient properties enabling it to be easily positioned around the desired body part. In other embodiments, as shown, the band 108 may have a clasp 116 or other coupling mechanism that enables the band 108 to be positioned around the desired body part.

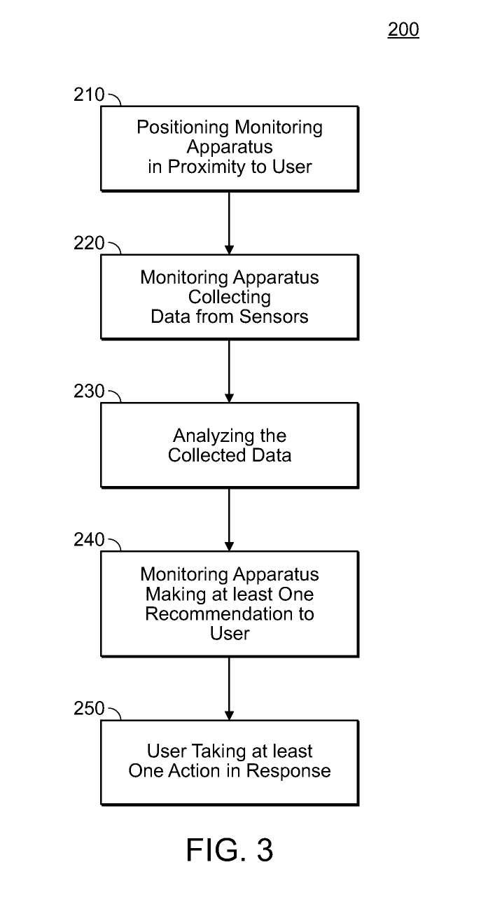

Referring now to FIG. 3, there is an overview of a method 200 associated with data collection and analyzation in accordance with an embodiment of the present invention.

In step 210, the monitoring apparatus is placed within a proximity to the human user. Typically, as described above, this entail placing the monitoring apparatus around a body part of the user such as an arm, wrist, neck, leg, torso, or the like. The coupling of the monitoring apparatus to the user enables various modules, such as a heartbeat module, to effectively monitor a human user's heartbeat. Further, the positioning allows other components such as the gyroscope and accelerometer to accurately and effectively measure the movements and positions of the human user.

In step 220, the monitoring apparatus begins collecting data from the user. In some instances, this data is input into the monitoring apparatus by the user and in other instances this data is measured by the monitoring apparatus itself. For example, the user may be required to input their height and weight into the monitoring apparatus. In turn, the monitoring apparatus may independently measure the user's heartbeat. The monitoring apparatus is worn for a predetermined amount of time to ensure a proper data set is collected. In some embodiments, this entails a user wearing the device constantly throughout the day and night.

In step 230, the collected data is analyzed. The collected data may comprise varying data points related to differing variables. For example, the data may be related to one's heartbeat throughout the day and night. A user may be able to input via the monitoring apparatus when they are exercising and resting such that the system can analyze and make recommendations accordingly. The data may be viewable from the monitoring apparatus or the electronic device. Further, the data collected is preferably stored on the server as described in FIG. 1. This data storage serves as a backup of the data as well as enables access to the data from anywhere in the world that has a suitable communication network connection.

In step 240, the monitoring apparatus and/or electronic device supplies at least one recommendation to the human user. Such a recommendation is based off the collected and analyzed data in steps 220 and 230. The monitoring apparatus sends the data to the electronic device and server, as noted, where it is analyzed by the programming or web application or mobile application or the like running thereon.

Recommendations are then made which can be pushed to the user via the monitoring apparatus and/or electronic device. In some instances, the recommendation is in the form of an email whereas in others it is a text message, SMS, MMS, or the like, or a combination thereof. The monitoring apparatus or electronic device may emit a sound, vibrate, emit lights, or the like to alert a user to the arrival of a recommendation from the system. For example, a user may receive a “chime” sound on their monitoring apparatus, and at least one module of the monitoring apparatus may vibrate in conjunction with the “chime.” When the user looks at the monitoring apparatus, they may view the notification automatically on the display or may interact with a touch sensitive button to receive/view the notification. A notification may read on either the electronic device or monitoring apparatus to remind a user to put on their light attenuating eyewear due to the time of day or may encourage them to exercise based on their perceived activity level. These notifications are intended to only be exemplary and any number or type of notifications may be received by the user.

In step 250, the user takes at least one action in response to the recommendation provided in step 240. Preferably, this action is in compliance with the recommendation in order to properly adjust or otherwise maintain their circadian or other biological cycle in a desirable rhythm.

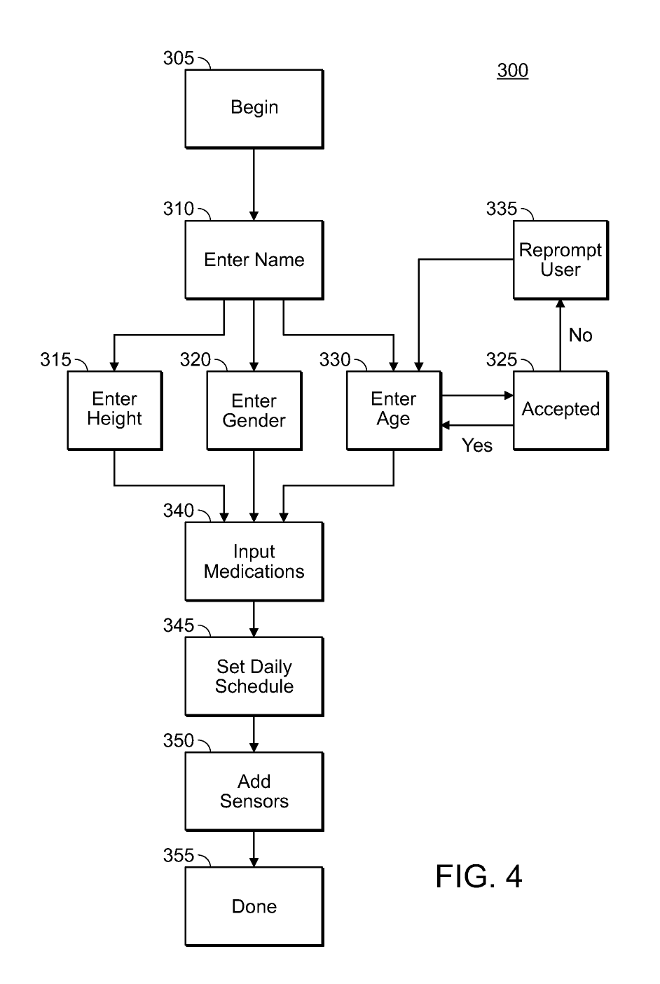

In FIG. 4, there is a flowchart describing a process to set up or initiate activity with the monitoring apparatus and/or electronic device. In order for the system as a whole to make the proper recommendations, some user input information, is required in order to ensure certain variables are taken into account. The flowchart contained herein has some of such variables which may be more or less than is actually required in order to achieve satisfactory results.

The method 300 generally begins in box 305, where the system determines that this is the user's first time accessing the system. A prompt may be displayed to the user asking them to sign in with their credentials or otherwise create a profile and credentials. In some embodiments, this can be done on both the monitoring apparatus and the electronic device, however, it may be preferable to have such steps completed on the electronic device.

In step 310, a user enters their name. The name entered may be a legal birth name, alias, nickname, or the like. In some instances, it may be preferable to use one's legal name in the event an outside source would like to examine the collected data.

In steps 315, 320, and 330 other general information is collected about such as a user's gender, age, weight, location, address, eye color, hair color, skin color, and the like or varying combinations thereof. As shown in box 325, in some instances, a check may occur to ensure a user did not input an improper or unreadable value that is outside of the parameters set by the system. For example, if a user were to input a numeral in the “name” field, the user may be prompted to reenter their name information as shown in box 335. Any input field may be subjected to this re-prompting process to ensure the correct information is obtained from the user thereby allowing the system to supply the correct recommendations.

In step 340, a user may input current medications to which they are prescribed or otherwise taking as part of a daily regimen. This allows the system to understand which medication(s) are being consumed, in which quantity, at what time, and if the medication(s) should be taken with meals or fluids. For example, the system may recommend that a user delay or defer the taking of a medication that may have a soporific side effect until the evening hours. Further, optimal timing for any and all medications may be made depending on the user's specific situation. In some instances, educational videos may be used in conjunction with the medication input and/or recommendations to encourage a user to take the medication at the appropriate time.

In step 345, a user may set their daily schedule. This may include but is not limited to times when a user wakes, eats, exercises, naps, and the like. These variables again provide the system with information that is used to make the best recommendations possible.

In step 350, a user may input any and all modules or sensors which they intend to use in conjunction with the monitoring apparatus. In other embodiments, the system may automatically ascertain which sensors or modules are being used thereby removing the need for this particular step. Once the user has entered the appropriate sensors, the process ends in box 355.

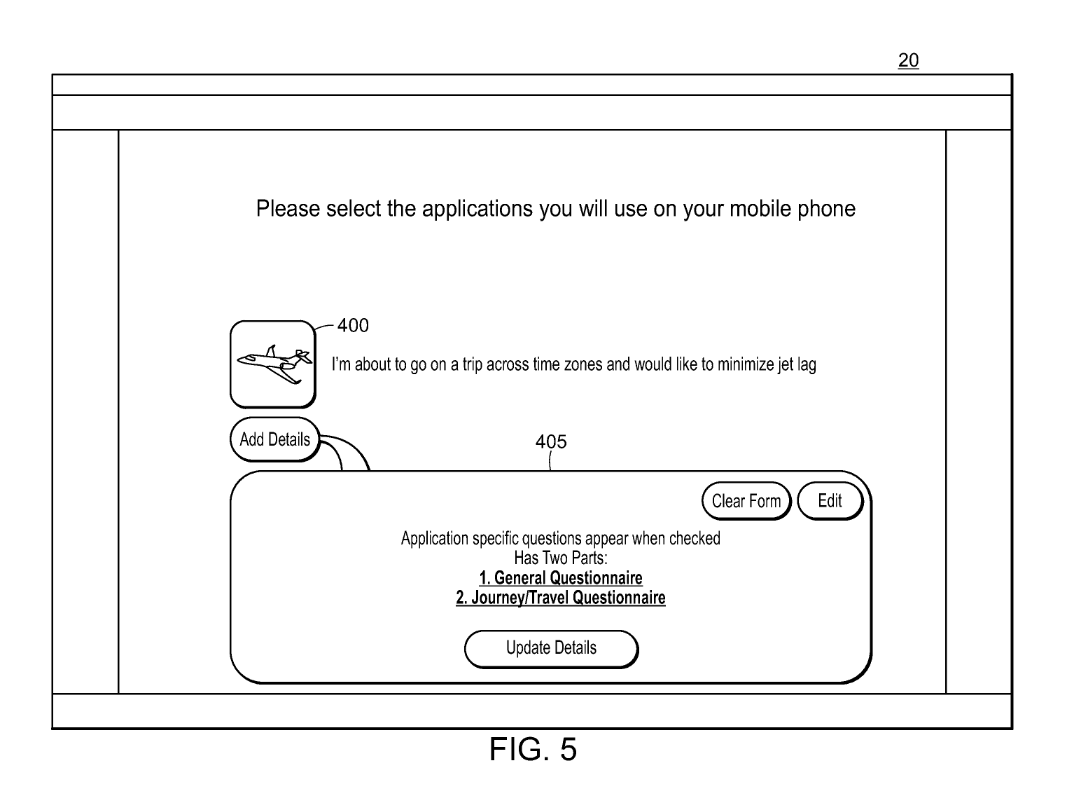

Referring now to FIGS. 5-7, a limited number of exemplary screens of an embodiment of the web/mobile application with which the electronic device and monitoring apparatus interface is shown.

In FIG. 5, there is an introduction or selection screen viewable on the display of an electronic device 20. The selection screen may enable a user to select and mix/match which applications the user would like to activate for use with the present system. The exact nature of the number of applications and their purpose may vary, however, some applications may include a jet lag application, circadian tuning application, breast cancer diagnostics application, and circadian tuning application for individuals with cancer or other ailments that may affect their biorhythms. While each application serves its own individual purposes the overall applications are tied together in that each preferably makes recommendations for tuning or adjusting one's biorhythms.

For example, a user may select a “jet lag” application that enables one to minimize the effects of jet lag by tuning their own naturally occurring biorhythms. The jet lag application icon 400 may be selected by the user. An application components box 405 may appear illustrating to a user the steps required to set up or initiate use of the application. In this example, there is both a “general questionnaire” and a “journey/travel questionnaire” for the user to complete before the application may function correctly.

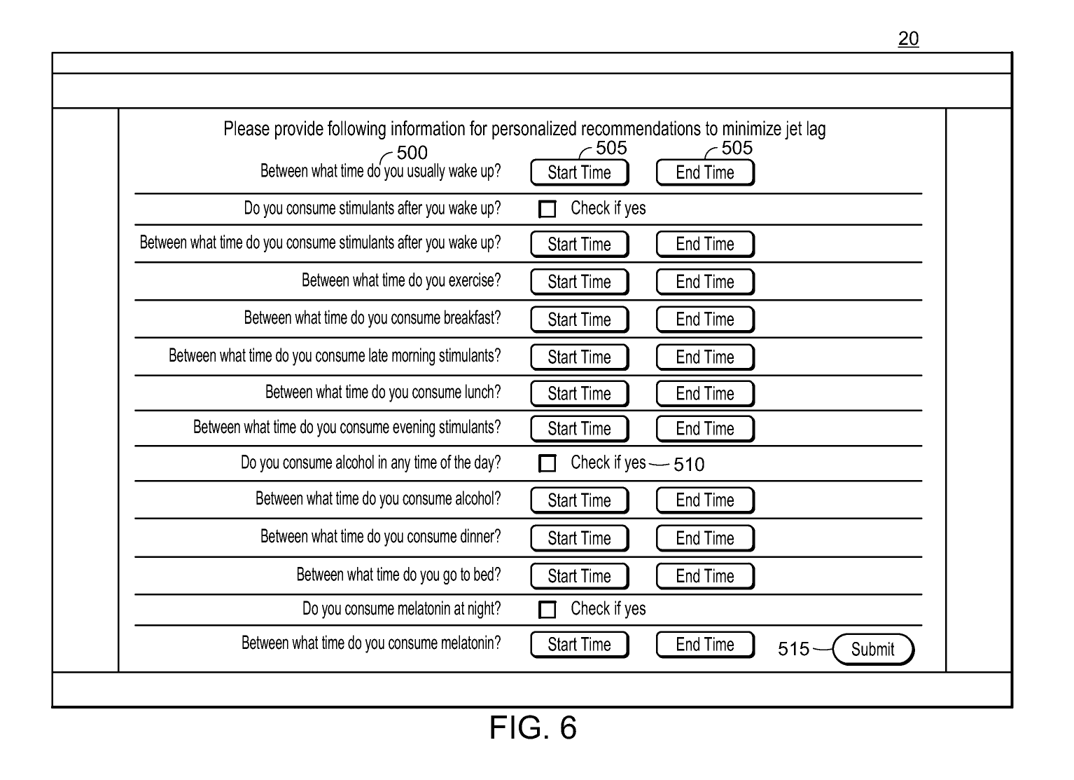

In FIG. 6, there is an example of a questionnaire associated with an application. Here, there are a number of questions 500 for a user to provide an answer. In some embodiments some questions may be optional whereas in others all questions may require an answer. Examples of questions be to posed to a user may be “between what time(s) do you consume breakfast” and “between what time(s) do you go to bed.” The user can then use selection buttons 505 to input an answer. In some embodiments the selection button 505 upon selection will open an input field for manual answer entry. In other instances a check box 510 may exist enabling someone to answer in the affirmative or to signify a particular value. Other embodiments regarding answer entry may exist including voice entry, manual selection, automatic selection (based on previously supplied information), and the like. Once a user has completed the questionnaire or a section thereof they may use a navigational button 515 to submit the answers or move to the next section.

In FIG. 7, there is a report screen as viewed on an electronic device 20 in some embodiments. The report screen may include various findings, recommendations, suggestions, alerts, and the like or a combination thereof. Here, for example, are findings 600 and recommendations 605 based on the information supplied by the user and the circadian rhythm engine which is capable of calculating an index of circadian entrainment. The findings 600 may include items such as a recitation of facts, statements based on answers, observations and/or analysis of one's circadian rhythm, and the like.

Recommendations 605 based on the findings and calculations by the circadian/biological rhythm engine provides recommendations to change or tune one's biological or circadian rhythm as well as recommendations to manage or maintain one's current biological or circadian rhythm. Such recommendations may include but are not limited to what time one should go to bed, what time one should arise in the morning, what time one should take their medication, what time one should eat, what type of food one should or should not eat or consume, what activities one should or should not partake, when to don one's light attenuating eyewear, what temperatures one should subject themselves, and the like and varying combinations thereof.

For example, alcohol and caffeine, depressants and stimulants respectively, should be taken at the appropriate times during one's circadian rhythm. Typically, stimulants should be taken in the early or late morning hours. Alcohol may be recommended to be taken in the later afternoon or early evening to avoid interference with REM sleep. However, the quantity consumed may also be as important, if not more important, than the timing of the consumption. There is empirical evidence to suggest that one or two alcoholic drinks may allow a person to achieve a deep sleep without affecting REM sleep. However, having more than two such drinks may have a detrimental effect on REM sleep. Thus, timing and quantity may go and be recommended hand in hand.

The recommendations may be pushed at once or intermittently throughout the day/night when most appropriate for the user. The recommendations may take the form of alerts or notifications that appear on the electronic device or monitoring apparatus or a combination thereof.

In general, the system and apparatus described herein are applicable for tuning and/or maintaining one's biological rhythms including their circadian rhythm to promote a healthy lifestyle or a higher quality of life for those who are ill.

For example, the system and apparatus may be used to implement a web or mobile application that prevents cancer through circadian rhythm tuning. Empirical evidence shows that environmental or genetic circadian disruption causes and accelerates cancer. By monitoring circadian sleep/activity and ambient light the system can provide precise real time measurement of the quality of day to day temporal organization. Thus, the system and application feeding back instructions for optimally timed riskless, costless behavioral environmental and melatonin-based, amongst other modifications, will re-align individuals' daily time structures. This natural alignment will allow them to feel better and function better and will diminish their risk for cancer.

Further, circadian rhythm tuning can be applied to breast cancer diagnostics, biopsy, and surgical breast cancer resection as it relates to the human menstrual cycle. In one embodiment, a user records their own menstrual cycle in real time for one or two cycles or more cycles in a mobile/web application that will allow analysis of when in the next cycle a screening mammogram can be scheduled to diminish the chances of missing a breast cancer. This is largely due to the fact that the denser the breast, the worse the sensitivity and specificity of any mammography for detecting breast cancer.

The most practical time span for receiving a mammography is located from about day one of the menstrual period to about at least one week thereafter. Such a mammographic scheduling will be both most revealing and most comfortable for the user since this is the time that monthly breast swelling and tenderness is diminishing most rapidly. Further, this time frame is the time in the cycle when the ovarian follicle is growing most rapidly, thereby referred to as the “follicular phase” of that cycle.

Additionally, a biopsy, if needed, can be done at an opposing time in the cycle which ranges from about fourteen (14) to about twenty-one (21) days after the first day of the last menstrual period. During this time period, any surgical intervention in the breast is less likely to support cancer growth and spread than in the “peri-menstrual phase.” This time frame is often referred to as the “luteal phase” of the menstrual cycle, in which little cell division is ongoing in the follicle. The follicle, at this stage, is referred to as the corpus luteum and it secretes many powerful hormones during this stage including, but not limited to, high concentrations of estrogens and progesterones. These high levels of hormones are a contributing factor to cause adverse cancer outcomes if a wounding of the biopsy occurs in their presence. According to empirical evidence, any surgical resection is about 26% more likely to be curative if it is perfumed at this luteal phase of the menstrual cycle.

Even still, if a user desires to protect her fertility and fecundity during any curative adjuvant chemotherapy, the user could schedule the chemotherapy during this luteal phase of the cycle when less cell division is occurring in the ovary. These factors and calculations allow the user to be in control of their medical treatments. The user can perform the scheduling of their mammography, biopsy, surgery, chemotherapy, and the like in accordance with their natural cyclic biology (i.e. biological rhythms).

In other instances, the circadian rhythms of those who already have some ailment, such as advanced metastatic cancer, can be assisted via this system to help feel them better, function better and live better longer.

Although this invention has been described with a certain degree of particularity, it is to be understood that the present disclosure has been made only by way of illustration and that numerous changes in the details of construction and arrangement of parts may be resorted to without departing from the spirit and the scope of the invention.

What is claimed is:

1. A system for modifying at least one biological rhythm of an individual, the system comprising:
at least one monitoring apparatus capable of monitoring at least one biorhythmic process,
wherein the at least one monitoring apparatus has an accelerometer and an ambient light sensor;
an electronic device capable of communicating with the at least one monitoring unit,
wherein the electronic device has a processor, a memory, and instructions for executing at least one program contained thereon,
wherein the at least one program analyzes data from the monitoring unit to calculate an index of circadian entrainment,
wherein the index of circadian entrainment is used to present feedback to a user.

2. The system of claim 1 further comprising a pair of eyeglasses that selectively filters light by wavelength.

3. The system of claim 2 wherein the pair of eyeglasses attenuates light having a wavelength of about 400 nm to about 500 nm.

4. The system of claim 1 wherein the at least one electronic device is capable of operating a web and/or mobile application thereon.

5. The system of claim 1 wherein the at least one monitoring apparatus further comprises at least one touch sensitive button.

6. The system of claim 5 wherein the at least one touch sensitive button enables tracking of time.

7. A system for modifying at least one biological rhythm of an individual, the system comprising:
monitoring apparatus comprising,
at least one sensing module, the at least one sensing module being capable of sensing ambient light, acceleration, and/or gravitational forces,
at least one power source operably coupled to the at least one sensing module,
a display module capable of displaying data relating to at least the one sensing module and/or at least one power source,
wherein the display module has a touch sensitive interface,
a motor unit disposed within either the display module or any of the at least one sensing modules, and
at least one light source; and
electronic device capable of communicating with the at least one monitoring unit,
wherein the electronic device has a processor, a memory, and instructions for executing at least one program contained thereon,
wherein the at least one program analyzes data from the monitoring unit to calculate an index of circadian entrainment,
wherein the index of circadian entrainment is used to present feedback to a user.

8. The system of claim 7 wherein the monitoring apparatus further comprises a wireless transceiver.

9. The system of claim 7 further comprising a pair of eyeglasses that attenuates light having a wavelength of about 400 nm to about 500 nm.

10. The system of claim 8 wherein the wireless transceiver operates in the 2.4 GHz industrial, scientific, and medical radio band.

11. A system for modifying at least one biological rhythm of an individual, the system comprising:
at least one sensor capable of measuring a physiological output of at least one user when kept in a proximity to the at least one user;
a wireless transceiver that transmits data associated with the at least one sensor;
a memory that stores the data associated with the at least one sensor;
a control unit operably coupled to the at least one sensor, the wireless transceiver, and the memory,
wherein the control unit enables the at least one user to interact with the system.

12. The system of claim 11 wherein the at least one sensor is a heart rate monitor, accelerometer, photosensor, temperature sensor, sound sensor, gyroscope, bioimpedance sensor, or any combination thereof.

13. The system of claim 11 further comprising a processor having computer readable instruction stored thereon,
wherein the processor is operably coupled to the memory.

14. The system of claim 13 wherein the computer readable instructions enable the processor to make recommendations based on a physiological measurement.

15. The system of claim 11 further comprising a sound emitting device.

16. A method for tuning and/or modifying at least one biological rhythm of an individual, the method comprising:
at least one user having a monitoring apparatus located in a proximity to the at least one user;
the monitoring apparatus collecting data via a least one sensor in the monitoring apparatus,
wherein the data pertains to physiological and/or environmental variables related to the at least one user;
analyzing the data; and
the monitoring apparatus making at least one recommendation to the at least one user based on the analyzed data.

17. The method of claim 16 further comprising the step of:
the at least one user taking at least one action in response to the at least one recommendation.

18. The method of claim 16 wherein the at least one recommendation pertains to eating patterns and/or habits of the at least one user.

19. The method of claim 16 wherein the at least one recommendation pertains to a sleep schedule of the at least one user.

20. The method of claim 16 wherein the at least one recommendation pertains to exercise for the at least one user.

21. A method for tuning and/or modifying at least one biological rhythm of an individual, the method comprising:
positioning a plurality of sensors to obtain one or more physiological and/or environmental parameters;
collecting data via the plurality of sensors,
wherein the plurality of sensors enables collection of data attributable to the one or more physiological and/or environmental parameters related to the at least one user;
analyzing the data via a processor of the monitoring apparatus,
wherein known values are compared to the one or more physiological and/or environmental parameters; and
the monitoring apparatus making at least one recommendation to the at least one user based on the analyzed data.

22. The method of claim 21 wherein the operable connection between the monitoring apparatus and the at least one user limits noise attributed to movement of the monitoring apparatus independent of the at least one user.

23. The method of claim 21 further comprising the step of:
transmitting the analyzed data to an electronic device capable of communicating with the monitoring apparatus.

24. The method of claim 21 wherein the data collected via the plurality of sensors is collected for at least twenty four continuous hours.

25. The method of claim 23 wherein the electronic device is a lap top computer, desktop computer, multimedia player, gaming system, smart watch, smart phone, or any combination thereof.

26. The method of claim 21 further comprising the step of:
inputting into a monitoring apparatus associated with the plurality of sensors at least one user provided parameter.

27. The method of claim 26 wherein the at least one user provided parameter pertains to a menstrual cycle.

28. A computer program embodied in a non-transitory computer-readable medium comprising computer readable instructions, which when executed by a processor, cause the processor to perform a method of instructing and/or causing modification to a biological rhythm of an individual, the method comprising:
collecting data from a plurality of sensors,
wherein the data pertains to the physiological and/or environmental state in which the plurality of sensors is present;
analyzing via a processor the data collected by the plurality of sensors; and
the processor making at least one recommendation based on the performed analysis.
